(19) United States
(12) Patent Application Publication
(10) Pub. No.: US 2025/0345200 A1
(43) Pub. Date: Nov. 13, 2025

(54) Reusable Ostomy Container

(71) Applicant: JUNE MING CHEN, San Bruno, CA (US)

(72) Inventor: JUNE MING CHEN, San Bruno, CA (US)

(21) Appl. No.: 18/662,454

(22) Filed: May 13, 2024

(51) Int. Cl. A61F 5/449; A61F 5/443

(52) U.S. Cl. CPC A61F 5/449; A61F 5/443

(57) ABSTRACT

A reusable ostomy container comprises a main body and a tightening member. The main body is an elastic hollow case, having a first case portion, a second case portion, and a storage space formed between the first case portion and the second case portion. An opening formed on the first case portion penetrates the first case portion and communicated with the storage space. The tightening member has a first tightening unit and a second tightening unit, the first tightening unit and the second tightening unit each has a portion attached to the second case portion, and the elongation of the first tightening unit is greater than that of the second tightening unit. The ostomy container provided by the present invention can effectively solve the cost problems, cleaning problems and health problems caused by the disposable ostomy pouches in the past.

### BACKGROUND OF THE INVENTION

### 1. Technical Field

The present invention relates to a medical device, and more particularly to a reusable ostomy container.

### 2. Description of Related Art

Artificial ostomy surgery is a common abdominal procedure in the medical field. It involves creating an opening on the body surface to access internal organs or cavities for draining fluids, excreta, or other therapeutic purposes. This procedure is typically used for individuals who have lost normal excretory function due to disease, injury, or surgery.

To manage artificial stoma, patients typically use a device called an ostomy pouch. An ostomy pouch is a bag-like device that can be securely attached to the artificial stoma to collect excreta or bodily fluids, maintaining cleanliness and hygiene in the surrounding area. These pouches are usually made of soft, waterproof materials and have adjustable seals to ensure that the contents do not leak. Due to hygiene and safety considerations, ostomy pouches are often designed to be disposable, requiring regular cleaning and replacement with new ones.

Despite providing convenience and hygiene in managing artificial stomas, disposable ostomy pouches still have some drawbacks: (1) High cost: Disposable ostomy pouches need to be replaced regularly, which increases the long-term cost burden of treatment. For some patients, long-term use may result in economic hardship. (2) Environmental impact: Extensive use of disposable ostomy pouches generates a significant amount of solid waste, posing an environmental burden. These plastic bags are often not recyclable or reusable, leading to waste and pollution. (3) Skin allergies or discomfort: Some patients may experience allergic reactions or skin discomfort from the materials in disposable ostomy pouches, leading to discomfort and skin damage. (4) Difficult to clean: Ostomy pouches usually are thin bag structures that are prone to deformation, with a small capacity and edges that are difficult to clean, leading to the accumulation of excreta in hard-to-reach areas.

### BRIEF SUMMARY OF THE INVENTION

In view of the above, the primary objective of the present invention is to provide a reusable ostomy container, which can effectively solve the related problems caused by the frequent replacement of conventional disposable ostomy pouches.

In order to achieve the objective of the present invention, the present invention provides an ostomy container, comprising: a main body, which is an elastic hollow case, having a first case portion and a second case portion on opposite sides, and a storage space formed between the first case portion and the second case portion; wherein an opening formed on the first case portion penetrates the first case portion and communicated with the storage space; and a tightening member, having a first tightening unit and a second tightening unit; the first tightening unit and the second tightening unit each having a portion attached to the second case portion, and an elongation of the first tightening unit is greater than that of the second tightening unit.

In an embodiment, the main body defines a maximum thickness as a farthest distance between the first case portion and the second case portion, and the maximum thickness is preferably greater than 10 mm, more preferably between 30 mm and 50 mm.

In an embodiment, the main body further comprises a piece-liked adsorption member movably received in the storage space.

In an embodiment, the main body further comprises a ring-shaped adhesive layer surrounding the opening, and the adhesive layer comprises a hydrocolloid dressing material.

In an embodiment, inner and outer surfaces of each corner of the main body are curved.

In an embodiment, the first tightening unit is located above the second tightening unit, and the first tightening unit is parallel to the second tightening unit.

In an embodiment, a width of the tightening member is preferably greater than two thirds of a width of the main body, more preferably between two thirds and three quarters of the width of the main body.

In an embodiment, a height of a top of the first tightening unit is preferably between the heights of a top and a bottom of the opening.

In an embodiment, the tightening member further comprises a pant unit which combines the first tightening unit and the second tightening unit.

In an embodiment, the second tightening unit is a structure formed by overlapping a plurality of first tightening units.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a perspective view of a first preferred embodiment of the present invention;

FIG. 2 is a perspective view of the main body of the first preferred embodiment of the present invention;

FIG. 3 is a sectional schematic view of the first preferred embodiment of the present invention, showing the user using the ostomy container; and

FIG. 4 is a schematic view of the first preferred embodiment of the present invention, showing how to clean the ostomy container;

FIG. 5 is a perspective view of a second preferred embodiment of the present invention, showing the adjusting member including Velcro structures;

FIG. 6 is a perspective view of a third preferred embodiment of the present invention, showing the tightening member formed as a closed ring-shaped structure; and

FIG. 7 is a schematic view of a fourth preferred embodiment of the present invention, showing the tightening member including a pant unit.

### DETAILED DESCRIPTION OF THE INVENTION

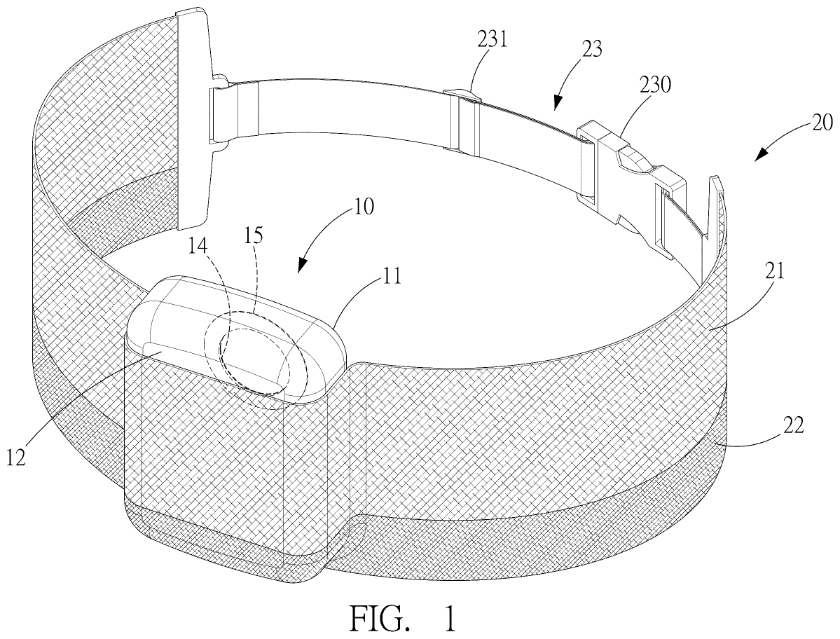

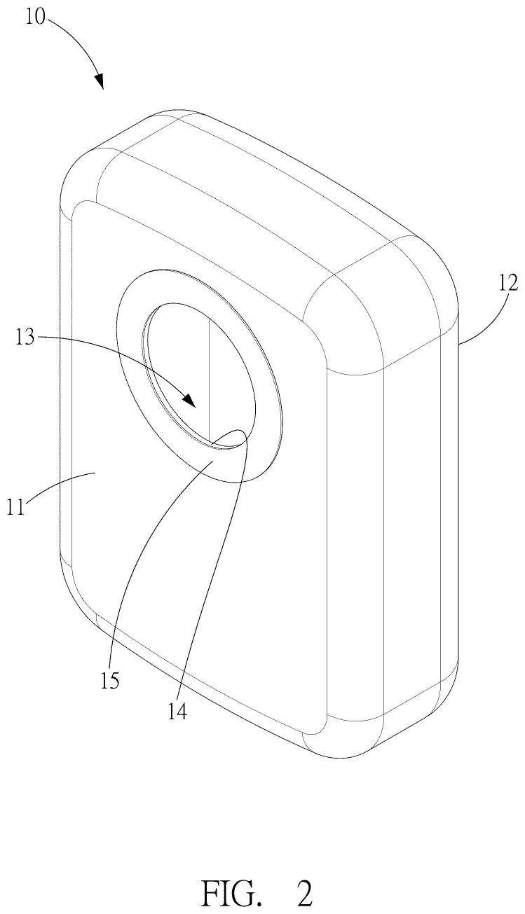

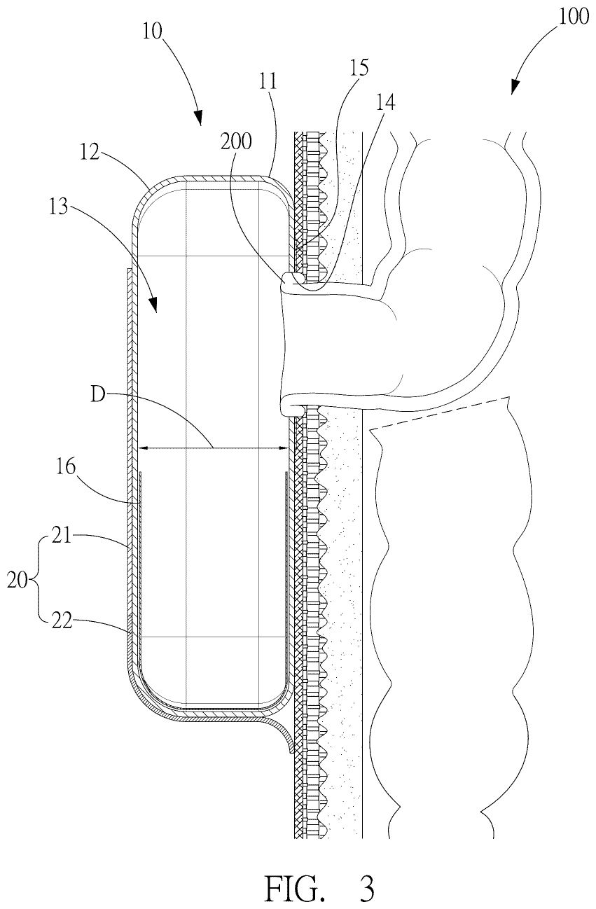

As shown in FIG. 1 to FIG. 3, an ostomy container of a first preferred embodiment of the present invention includes a main body 10 and a tightening member 20.

The main body 10 is a hollow case made of elastic material, having a first case portion 11 and a second case portion 12 on opposite sides. A storage space 13 is formed between the first case portion 11 and the second case portion 12. The main body 10 provided with an opening 14 passed through the first case portion 11 and communicated with the storage space 13. The opening 14 is located at an upper position of the first case portion 11, and the size of the opening 14 is greater than that of an artificial stoma 200 of a user 100. Thereby, when the main body 10 is rest against the user 100, the opening 14 may cover the artificial stoma 200 so that excreta discharged from the artificial stoma 200 passes through the opening 14 and is stored in the storage space 13.

Since the main body 10 is a hollow case, a maximum thickness D is defined as a farthest distance between the first case portion 11 and the second case portion 12. In the present preferred embodiment, the maximum thickness D is preferably greater than 10 mm, and more preferably between 30 mm and 50 mm.

In the present preferred embodiment, inner and outer surfaces of each corner of the main body 10 are generally curved.

In the present preferred embodiment, the main body 10 further includes a ring-shaped adhesive layer 15 formed around the opening 14. The adhesive layer 15 is made of a hydrocolloid dressing material and is configured to provide viscosity to adhere to the skin around the artificial stoma 200 and provide liquid-absorbing function.

In the present preferred embodiment, the main body 10 is made of silicon.

The tightening member 20 is attached to the main body 10, which is configured to secure the main body 10 on the user 100. In the present preferred embodiment, the tightening member 20 includes a first tightening unit 21 and a second tightening unit 22. The first tightening unit 21 is located above the second tightening unit 22. When the tightening member 20 is attached to the main body 10, the first tightening unit 21 covers an upper edge of the second case portion 12 of the main body 10, and the second tightening unit 22 covers a lower edge of the second case portion 12 of the main body 10. Wherein, the first tightening unit 21 and the second tightening unit 22 are elastic bands with different tightness, and an elongation of the first tightening unit 21 are greater than that of the second tightening unit 22. Wherein the so-called elongation refers to the elasticity of the elastic band itself, that is, the maximum stretchable length. The greater the elongation, the greater the degree of deformation of the elastic band. In other words, the first tightening unit 21 is an elastic band structure which has a greater degree of deformability; and the second tightening unit 22 is an elastic band structure which has smaller degree of deformability. Thereby, when the tightening member 20 is wrapped around a waist of the user 100, the first tightening unit 21 facilitates in the adjustment of the position of the main body 10, and the second tightening unit 22 facilitates in the support to the main body 10, preventing the main body 10 from falling.

In the present preferred embodiment, a width of the tightening member 20 is preferably greater than two thirds of a width of the main body 10, more preferably between two thirds and three quarters of the width of the main body 10. Thereby, a height of the top of the first tightening unit 21 is preferably between the top and the bottom of the opening 14, more preferably greater than the top of the opening 14, so that the first tightening unit 21 is able to force the opening 14 engaged with the user.

In the present preferred embodiment, the tightening member 20 is detachably attached to the main body 10. In an alternative preferred embodiment, the main body and the tightening member can form a non-detachable fixed state through gluing, sewing, snapping or any other fixing method.

In the present preferred embodiment, the first tightening unit 21 and the second tightening unit 22 are an integrally formed elastic band structure. In an alternative preferred embodiment, the first tightening unit and the second tightening unit are two separate elastic band structure.

In the present preferred embodiment, the tightening member 20 includes two elastic band structures such as the first tightening unit 21 and the second tightening unit 22. In an alternative preferred embodiment, the tightening member may include only one elastic band structure. It should be noted that the tightening member also can be formed as the first tightening unit including an elastic band and the second tightening unit including two overlapping elastic bands.

The tightening member further has an adjusting member 23 which is connected to both ends of the tightening member 20 and can be operated to adjust the length of the tightening member 20, so that the tightening member 20 can be adapted to users with different waist sizes. For example, in the present preferred embodiment, the adjusting member 23 includes a buckle 230 and an adjusting strap 231. The buckle 230 is configured to be operated to switch the tightening member 20 between a closed state and an open state. The adjustment strap 231 is configured to be operated to change the total length of the tightening member 20.

When the user 100 is going to use the ostomy container provided by the present invention, the user 100 faces the opening 14 of the body 10 toward the body direction and is aligned with the artificial stoma 200. Next, the user 100 fits the main body 10 to the waist, wraps the tightening member 20 around the waist, and adjusts the length of the tightening member 20 to an appropriate tightness through the adjusting member 23, and the installation of the ostomy container is completed. In the user's daily life, the main body 10 is like a general ostomy pouch, which can store the excrement discharged from the artificial stoma 200 through the storage space 13, and the adhesive layer 15 around the opening 14 facilitates the connection to the skin and prevents excrement from leaking out.

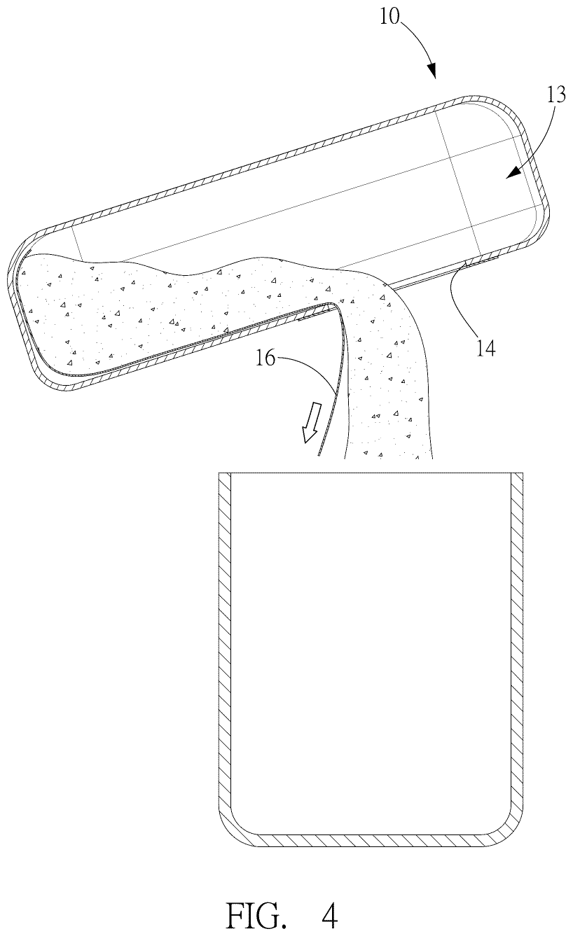

As shown in FIG. 4, the difference between the present invention and the disposable ostomy pouch is that when the excreta in the storage space 13 reaches a certain amount, the user 100 only needs to untie the tightening member 20 to separate it from the user 100, and the main body 10 can be removed. The user can dump the main body 10 so that the excreta inside the main body 10 can be discharged into a clean container (such as a toilet or a garbage bag) along the opening 14, and the storage space 13 of the main body 10 can be cleaned can be cleaned by water. At the same time, since the corners of the main body 10 are curved, the excreta can be more effectively removed from the storage space 13, thus enhancing the reusability of the main body 10. In addition, in the present preferred embodiment, the main body 10 is further provided with a pieced-like adsorption member 16 movably received in the main body 10 to absorb the excreta entering the storage space 13 and can be removed along the opening 14 during cleaning. Since the main body 10 is an elastic hollow case made of silicone, it can be easily cleaned with water or detergent and can be reused. Thereby, the ostomy container of the present invention can be reused, solving various problems of users when using disposable ostomy pouches.

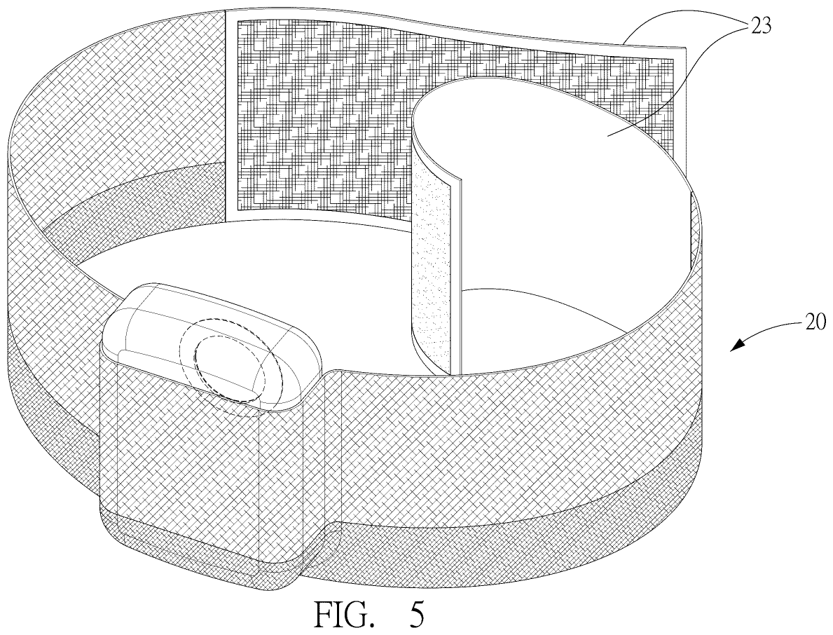

As shown in FIG. 5, an ostomy container of a second preferred embodiment of the present invention has a structure similar to that of the first preferred embodiment. In the present preferred embodiment, the adjusting member 23 of the tightening member 20 includes two Velcro structures mounted on opposite ends of the tightening member 20.

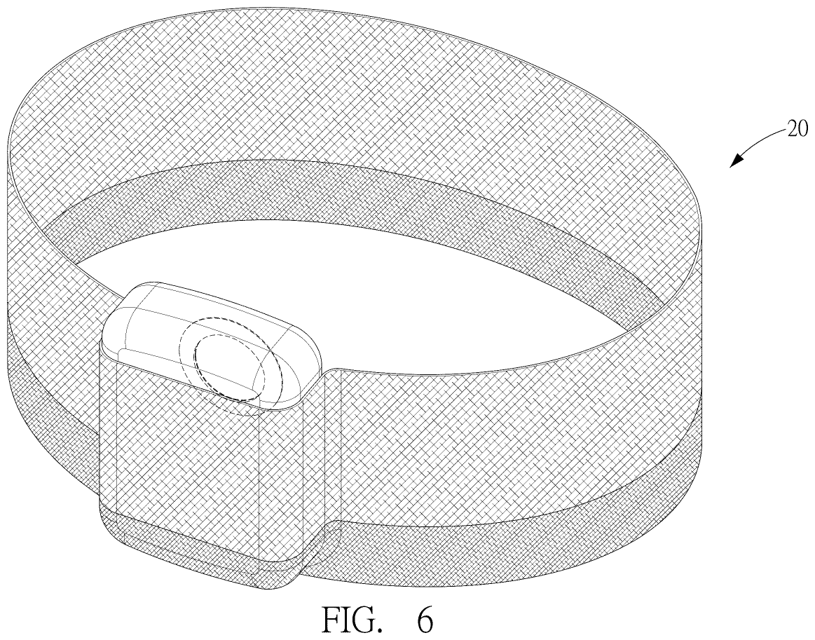

As shown in FIG. 6, an ostomy container of a third preferred embodiment of the present invention has a structure similar to that of the first preferred embodiment. In the present preferred embodiment, the tightening member 20 is formed as a closed ring-shaped band structure without any free end. Thereby the user may change the size of the tightening member 20 by stretching the tightening member 20.

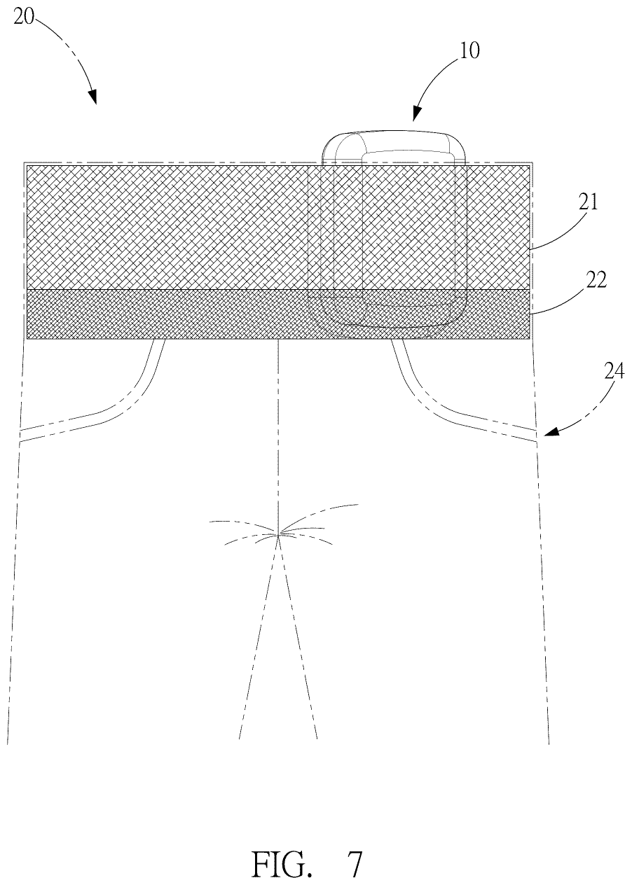

As shown in FIG. 7, an ostomy container of a fourth preferred embodiment of the present invention has a structure similar to that of the first preferred embodiment. In the present preferred embodiment, the tightening member 20 further includes a pant unit 24, which is in a shape of a short or a pant, and the first tightening unit 21 and the second tightening unit 22. When the user 100 equips the tightening member 20, the pant unit 24 can be worn on the lower body of the user 100 as a normal pant. The installation of the remaining parts is the same as the first preferred embodiment and will not be described again here. Through the structure of the pant unit 24, the main body 10 can be more to the user 100 can be enhanced.

In conclusion, the ostomy container provided by the present invention can effectively solve the cost problems, cleaning problems and health problems caused by the disposable ostomy pouches in the past by virtue of the reusable characteristics of the main body 10. On the other hand, through the different tightness of the first tightening unit 21 and the second tightening unit 22 of the tightening member 20, the fixed strength of the main body 10 can be maintained while conveniently adjusting its position, thereby improving the ostomy container experience.

It must be pointed out that the embodiments described above are only some preferred embodiments of the present invention. All equivalent structures which employ the concepts disclosed in this specification and the appended claims should fall within the scope of the present invention.

What is claimed is:

1. An ostomy container, comprising:
a main body, which is an elastic hollow case, having a first case portion and a second case portion on opposite sides, and a storage space formed between the first case portion and the second case portion; wherein an opening formed on the first case portion penetrates the first case portion and communicated with the storage space; and
a tightening member, having a first tightening unit and a second tightening unit; the first tightening unit and the second tightening unit each having a portion attached to the second case portion, and an elongation of the first tightening unit is greater than that of the second tightening unit.

2. The ostomy container of claim 1, wherein the main body defines a maximum thickness as a farthest distance between the first case portion and the second case portion, and the maximum thickness is preferably greater than 10 mm, more preferably between 30 mm and 50 mm.

3. The ostomy container of claim 1, wherein the main body further comprises a piece-liked adsorption member movably received in the storage space.

4. The ostomy container of claim 1, wherein the main body further comprises a ring-shaped adhesive layer surrounding the opening, and the adhesive layer comprises a hydrocolloid dressing material.

5. The ostomy container of claim 1, wherein inner and outer surfaces of each corner of the main body are curved.

6. The ostomy container of claim 1, wherein the first tightening unit is located above the second tightening unit, and the first tightening unit is parallel to the second tightening unit.

7. The ostomy container of claim 1, wherein a width of the tightening member is preferably greater than two thirds of a width of the main body, more preferably between two thirds and three quarters of the width of the main body.

8. The ostomy container of claim 1, wherein a height of a top of the first tightening unit is preferably between the heights of a top and a bottom of the opening.

9. The ostomy container of claim 1, wherein the tightening member further comprises a pant unit which combines the first tightening unit and the second tightening unit.

10. The ostomy container of claim 1, wherein the second tightening unit is a structure formed by overlapping a plurality of first tightening units.
